(19) United States
(12) Patent Application Publication
(10) Pub. No.: US 2004/0131544 A1
(43) Pub. Date: Jul. 8, 2004

(54) In vivo imaging

(76) Inventors: Derek MacLean, Mountain View, CA (US); Joshua M. Star-Lack, Palo Alto, CA (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/739,828

(22) Filed: Dec. 18, 2003

Related U.S. Application Data
(62) Division of application No. 09/693,410, filed on Oct. 20, 2000, now abandoned.
(60) Provisional application No. 60/160,918, filed on Oct. 22, 1999.

(51) Int. Cl. A61K 51/00; G01N 33/53

(52) U.S. Cl. 424/1.11; 435/7.1

(57) ABSTRACT

A positron emission tomography (PET)-ready library of candidate pharmaceutical agents is provided which can be prepared by a multistep process wherein the final or penultimate step is a reaction using a PET-ready reagent or a plurality of PET-ready reagents. Methods of preparing and using the libraries are also provided.

### CROSS-REFERENCES TO RELATED APPLICATIONS

This application claims the benefit of Provisional Application Serial No. 60/160,918, filed Oct. 22, 1999, the disclosure of which is incorporated herein by reference.

### BACKGROUND OF THE INVENTION

In the development of a therapeutic agent it is highly desirable to determine the agent's biodistribution in an animal of interest, typically a human. More particularly, drug development now often involves a determination of a drug's bioavailability, it's passage across the blood-brain-barrier, and distribution in various tissues. Typically, this data is collected using such invasive techniques as blood sampling and tissue dissection. The latter techniques are not amenable to humans and in vitro methods have been developed to model the desired in vivo studies. These in vitro techniques include MDCK permeability and in silico methods to model, for example, the blood-brain-barrier. Despite the initial utility of these methods, each represents only an approximation of an agent's behavior in vivo.

More direct methods to study an, agent's biodistribution in the body involve magnetic resonance imaging (MRI), positron emission tomography (PET), and single photon emission computed tomography (SPECT). Each of these methods can detect the distribution of a compound within the body if that compound contains an atom with the appropriate nuclear properties. MRI detects paramagnetic nuclei; PET and SPECT detect the emission of particles from the decay of radionuclei.

Most therapeutic agents are not able to be detected by these techniques without modification. Thus, for PET it is necessary to incorporate an appropriate positron-emitting radionuclide. There a relatively few positron-emitting isotopes which are suitable for labeling a therapeutic agent. The carbon isotope, ¹¹C, has been used for PET, but its short half-life of 20.5 minutes limits its usefulness to compounds that can be synthesized and purified quickly, and to facilities that are proximate to a cyclotron where the precursor ¹¹C starting material is generated. Other isotopes have even shorter half-lives. ¹³N has a half-life of 10 minutes and ¹⁵O has an even shorter half-life of 2 minutes. The emissions of both are more energetic than those of ¹¹C and PET studies have been carried out with these isotopes (see, CLINICAL POSITRON EMISSION TOMOGRAPHY, Mosby Year Book, 1992, K. F. Hubner, et al., Chapter 2). Another useful isotope, ¹⁸F, has a half-life of 110 minutes. This allows sufficient time for incorporation into a radiolabeled tracer, for purification and for administration into a human or animal subject. Use of ¹⁸F labeled compounds in PET has been limited to a few analog compounds. Most notably, ¹⁸F-fluorodeoxyglucose has been used in studies of glucose metabolism and localization of glucose uptake associated with brain activity. ¹⁸F-L-fluorodopa and other dopamine receptor analogs have also been used in mapping dopamine receptor distribution.

SPECT imaging employs isotope tracers that emit high energy photons (γ-emitters). The range of useful isotopes is greater than for PET, but SPECT provides lower three-dimensional resolution. Nevertheless, SPECT is widely used to obtain clinically significant information about analog binding, localization and clearance rates. A useful isotope for SPECT imaging is ¹²³I, a γ-emitter with a 13.3 hour half life. Compounds labeled with ¹²³I can be shipped up to about 1000 miles from the manufacturing site, or the isotope itself can be transported for on-site synthesis. Eighty-five percent of the isotope's emissions are 159 KeV photons, which is readily measured by SPECT instrumentation currently in use.

Other halogen isotopes can serve for PET or SPECT imaging, or for conventional tracer labeling. These include ⁷⁵Br, ⁷⁶Br, ⁷⁷Br and ⁸²Br as having usable half-lives and emission characteristics. In general, the chemical means exist to substitute any halogen moiety for the described isotopes. Therefore, the biochemical or physiological activities of any halogenated homolog of the described compounds are now available for use by those skilled in the art, including stable isotope halogen homologs.

A common approach to biodistribution studies using PET or SPECT involves modifying an existing therapeutic agent or drug candidate to incorporate an appropriate atom for the selected imaging modality. For example, an active agent may be modified to incorporate a fluorine or iodine isotope with desirable imaging properties. While the derivative produced can be detected, other properties of the compound (e.g., electronic properties leading to enhanced reactivity, or steric properties which hamper a compound's binding to a target) may have been altered, rendering the biodistribution studies of limited value. In some instances, the modification of an existing therapeutic agent can result in a derivative having significant adverse properties. For example, a fluorinated derivative of carazolol was found to be mutagenic, while the parent compound was not toxic (see Doze, et al., Nuclear Medicine and Biology 27:315-319 (2000)).

An alternative approach involves replacing one atom of the therapeutic agent or candidate drug with a different isotope of the same atom. In this manner, the agent or candidate drug is chemically identical to the initial agent or drug. As noted above, PET is one of the most useful imaging techniques, but the half-lives of the positron-emitting isotopes of the elements which are commonly found in drugs (carbon, nitrogen, oxygen, and less commonly fluorine) are extremely short (10 min to 2 h). This causes considerable technical difficulties in incorporating the isotope and preparing the therapeutic agent before the isotope has substantially decayed. It would be helpful if the candidate drug or agent was prepared using methods which could be suitably modified for incorporation of, for example, a PET label. Unfortunately, many existing drugs or lead compounds are not amenable to this approach as they were not designed with due consideration for imaging during development. In particular, the chemical route used to prepare these therapeutic agents does not permit introduction of such an isotope in the final stages of synthesis, to allow the agent to be prepared and formulated before substantial radioactive decay. Combinatorial library technology offers the ability to prepare large numbers of compounds with a defined chemical route which may be designed to permit ready introduction of such an isotope. What is needed in the art are libraries of candidate therapeutic agents that can be suitably derivatized to incorporate a label in the final stages of synthesis.

The present invention fulfills this and other needs.

### SUMMARY OF THE INVENTION

In one aspect, the present invention provides a positron emission tomography (PET)-ready library of candidate pharmaceutical agents. The library is prepared by a multistep process in which the final or penultimate step is a reaction using a PET-ready reagent or a plurality of PET-ready reagents. In one group of embodiments, each member of the nascent library is treated with the same PET-ready reagent. In another group of embodiments, each member of the nascent library is treated with a plurality of PET-ready reagents. The libraries of the present invention will typically have from 10 to 100,000 members, but may have from 100,000 to 1,000,000 members or more.

In another aspect; the present invention provides methods of preparing a positron emission tomography (PET)-ready library of candidate pharmaceutical agents. In general, the methods provide treating a library of compounds with a PET-ready reagent or a plurality of PET-ready reagents to produce a PET-ready library of candidate pharmaceutical agents in which each member of the library has been exposed to and preferably has reacted with a PET-ready reagent. In one group of embodiments, the PET-ready library is prepared in solution. In another group of embodiments, the PET-ready library is prepared on a solid support (e.g., a resin, a glass slide or a bead). In yet another group of embodiments, the PET-ready library is a library in which each member is “tagged” for identification.

In yet another aspect, the present invention provides a method for determining the distribution of an active agent in a tissue, comprising:

(a) screening a PET-ready library of candidate pharmaceutical agents against a biological target;

(b) identifying at least one of the candidate pharmaceutical agents as an active agent;

(c) preparing a PET-labeled version of the active agent, wherein the preparing comprises incorporating a PET-label into the final or penultimate step of active agent synthesis;

(d) administering the PET-labeled version of the active agent to a subject; and

(e) measuring the distribution of the active agent in at least one tissue of the subject.

In still another aspect, the present invention provides reagents and methods for the preparation of PET-ready libraries or individual PET-labeled or PET-ready compounds.

In yet another aspect, the present invention provides a method for preparing a PET-labeled compound, the method comprising:

(a) providing a precursor compound covalently attached to a solid support;

(b) contacting said precursor compound with a PET-labeled reagent to produce a composition comprising a PET-labeled compound portion attached to said solid support by a linking group; and

(c) removing said PET-labeled compound from said composition under conditions whereby any unreacted precursor compound remains covalently attached to said solid support.

In a related embodiment, the PET-labeled compound is prepared and removed from the solid support under conditions which favor product removal over removal of the starting material.

### DETAILED DESCRIPTION OF THE INVENTION

The present invention provides libraries of candidate agents for pharmaceutical screening that are designed to allow the incorporation of a PET-label in the final or penultimate step of synthesis. In addition to the libraries, the present invention provides methods for preparing the libraries and methods of using the libraries.

As noted above, the advent of combinatorial chemistry and high-throughput screening has hastened the development of candidate pharmaceutical agents. More specifically, the noted processes have greatly reduced the amount of time to discover a “lead” compound. Nevertheless, significant research takes place once a lead compound is discovered. This research typically takes the form of determining structure-activity relationships in derivative compounds that are prepared based on a lead structure. While the development of these relationships can lead to more potent compounds in various in vitro and in vivo assays, other critical parameters for the compounds are often neglected, typically until much later in the development process. Among the most important among these parameters is a tissue distribution profile for the derivative compound.

The development of a tissue distribution profile is often ignored until late in the drug development process. At this point, the synthesis of a candidate pharmaceutical agent is often well-characterized and not readily refined or altered, making the incorporation of a radiolabel a challenging hurdle.

The present invention provides a method of preparing a library of compounds which can be readily altered to introduce a label, typically a PET label. While the invention is described below for the development of PET imaging agents, one of skill in the art will appreciate that SPECT and/or MRI imaging agents can be obtained by similar approaches. In brief, the methods and libraries provided herein, are those methods in which a label can be introduced in the final or penultimate step of synthesis.

PET-Ready Libraries

Primary chemical compound libraries for general screening offer a unique advantage for PET-ready compound development as the synthesis route for the library members is often designed de novo. Accordingly, the route can be set up or designed to provide a final or penultimate step that can incorporate an atom having an isotope with good PET properties. The term “PET-ready” when used to refer to a particular reagent, compound or library, refers to a “cold” reagent, compound or library that is the chemical equivalent of a PET-labeled version. For example, a “PET-ready reagent” is a chemical reagent that is readily available from sources such as Aldrich Chemical Company and other suppliers in “cold” form and can be readily prepared as a labeled version (e.g., CH₃I and ¹¹C—CH₃I, F₂ and ¹⁸F—F, KF and K¹⁸F, CH₃COCl and ¹¹C—CH₃COCl, and the like).

A “PET-ready compound” or “PET-ready agent” is similarly a compound or agent (typically a member of a library of compounds or agents) that can be prepared in a labeled form without alteration of its chemical structure. For example, fluorodeoxyglucose is a “PET-ready agent,” with ¹⁸F-fluorodeoxyglucose being the PET-labeled version thereof. A “PET-ready library,” as described in more detail below, is a library of chemical compounds or candidate pharmaceutical agents which, by their design, can be prepared in a PET-labeled version. Typically, at least about 50% of the members of a PET-ready library can be prepared in a PET-labeled form without altering the chemical structure of the individual agent or compound. Preferably at least about 70%, more preferably at least about 80% and most preferably at least about 90% of the PET-ready library members can be prepared in a labeled form without altering the chemical structure of the compound.

Thus, in one aspect, the present invention provides a positron emission tomography (PET)-ready library of candidate pharmaceutical agents. As used herein, a chemical or combinatorial “library” is an intentionally created collection of differing molecules which can be prepared by the synthetic means provided below or otherwise and screened for biological activity in a variety of formats (e.g., libraries of soluble molecules, libraries of compounds attached to resin beads, silica chips or other solid supports). Additionally, the term “combinatorial chemistry” or “combinatorial synthesis” refers to the synthesis of diverse compounds by sequential addition of reagents or PET-ready reagents which leads to the generation of large chemical libraries having molecular diversity. Combinatorial chemistry, therefore, involves the systematic and repetitive, covalent connection of a set of different “building blocks” of varying structures to yield large arrays of diverse molecular entities.

Additionally, the libraries will preferably have from about 12 to about 100,000 members or more. More preferably, the libraries will have from about 12 to about 50,000 members. Most preferably, the libraries will have from about 12 to about 96 members.

The libraries of the present invention preferably have at least one active compound and are prepared in a manner to provide the library members in approximately equimolar quantities. It should be appreciated, however, that such libraries can comprise several smaller “sub-libraries” or sets of compounds or sets of mixtures of compounds, depending on the format of preparation and the varying groups that are attached to a central “core structure” or “scaffold.”

As just noted, the PET-ready libraries of candidate pharmaceutical agents can have a variety of core structures or scaffolds on which the libraries are built. For example, the libraries can have a core structure that is a carbohydrate, an amino acid, an aromatic or heteroaromatic ring (e.g., phenyl, naphthyl, quinoline, quinoxaline and the like), a heterocyclic ring, a nucleic acid (typically in the form of a purine or pyrimidine core), and combinations thereof. Common to the selection of a core structure or scaffold, however, is the ability to create diversity in the library by reacting and derivatizing any of two or more reactive centers. See, for example, U.S. Pat. No. 5,948,696 (“Combinatorial biaryl amino acid amide libraries”); U.S. Pat. No. 5,942,387 (“Combinatorial process for preparing substituted thiophene libraries”); U.S. Pat. No. 5,925,527 (“Tricyclic tetrahydroquinoline derivatives and tricyclic tetrahydroquinoline combinatorial libraries”); U.S. Pat. No. 5,859,190 (“Combinatorial libraries of hydantoin and thiohydantoin derivatives, methods of making the libraries and compounds therein”); U.S. Pat. No. 5,840,500 (“Quinoline derivatives and quinoline combinatorial libraries”); U.S. Pat. No. 5,821,130 (“Combinatorial dihydrobenzopyran library”); U.S. Pat. No. 5,783,577 (“Synthesis of quinazolinone libraries and derivatives thereof”); U.S. Pat. No. 5,618,825 (“Combinatorial sulfonamide library”); U.S. Pat. No. 5,569,588 (Isoprenoid libraries); U.S. Pat. No. 5,549,974 (Metathiazinone libraries); U.S. Pat. No. 5,525,734 (“Methods for synthesizing diverse collections of pyrrolidine compounds”); U.S. Pat. No. 5,506,337 (Morpholino compound libraries) and U.S. Pat. No. 5,288,514 (“Solid phase and combinatorial synthesis of benzodiazepine compounds on a solid support”). See also, WO 96/00391 (“Method for the synthesis of diketopiperazines”).

One of skill in the art will understand that any of the methods in the above patents and PCT publication (as well as other methods readily available to the practitioner) can be used to initiate synthesis of the present libraries. However, the developing or nascent libraries will be further derivatized with a PET-ready reagent or a plurality of PET-ready reagents to produce a PET-ready library. This design and construction of library members will facilitate incorporation of a PET-label once a suitably active compound is identified. Accordingly, the libraries are those which can be prepared by a multi-step process wherein the final or penultimate step of the multistep process is a reaction in which a PET-ready reagent or a plurality of PET-ready reagents (“cold” forms of PET-labeled reagents) is used. The term “final or penultimate step” refers to a discrete chemical reaction in a synthesis route and does not include steps such as isolation, purification (e.g., chromatography, crystallization, filtration, and the like) or cleavage from a support. Typically the reactions used in the final or penultimate step are those reactions in which a new bond is formed between, for example, two carbon atoms, carbon and halogen, carbon and nitrogen, carbon and oxygen, or carbon and sulfur. Additionally, the reaction step referred to is typically one which produces an isolable compound or intermediate (whether or not the intermediate is actually isolated). For example, a final or penultimate step can be a reaction such as an alkylation reaction, an acylation reaction, a carbonylation reaction, a Wittig-type reaction, a Diels-Alder reaction, a reductive amination reaction, an aromatic substitution reaction, a halogen exchange reaction, nucleophile substitution, electrophilic substitution, oxidation, and a reduction reaction. In some embodiments, compounds are formed in a single reaction involving multiple reagents (e.g., the Ugi reaction). In such instances, the final or penultimate step refers to the multistep process wherein one of the reagents can be a PET-ready reagent.

As noted above, the PET-ready library is designed to allow facile labeling with PET labels once an active compound is identified. Suitable labels include, for example, ¹¹C, ¹⁸F, ¹³N, ⁷⁶Br, ¹⁵O, ¹²⁴I, and the like. In general, the PET label is a label which is covalently attached to the remainder of the molecule and should have a half-life of at least about 5 minutes, preferably about 10 to 20 minutes or more. Particularly preferred PET labels for consideration in design of the PET-ready library are ¹¹C, ¹⁸F, ¹³N, ⁷⁶Br and ¹²⁴I.

Carbon-11 (¹¹C) is a positron-emitting (99+%) radionuclide that decays with a half-life of 20.4 minutes to a stable nuclide, boron-11, with emission of a high-energy positron (Eₘₐₓ=0.96 MeV). This radionuclide is produced in the chemical form of carbon dioxide by proton irradiation of nitrogen-14 gas. ¹¹C-carbon dioxide can be readily converted to a variety of reagents including ¹¹C-carbon monoxide, ¹¹C-phosgene, ¹¹C-acetyl chloride, ¹¹C-methyl iodide, ¹¹C-methyl triflate, ¹¹C-cyanogen bromide and ¹¹C-methyl lithium, providing synthesis avenues into a numerous PET-labeled compounds. Still other ¹¹C-labeled reagents that are available include: ¹¹CH₂N₂, ¹¹CH₃NCO, ¹¹CH₃NO₂, (R)₃P⁺¹¹CH₃I, H¹¹CN, R¹¹CH₂OH, R¹¹CH₂I, R¹¹CH₂NO₂, R¹¹CH₂NCO, R¹¹CHO, and the like (see, PRINCIPLES OF NUCLEAR MEDICINE, 2_{ND} ED. pp. 166-178, (1995)).

Similarly, fluorine-18 (¹⁸F) is another useful positron-emitting radionuclide. Its half-life of 110 minutes permits its use in synthesis procedures and imaging methods that can extend over periods of several hours. Reagents useful for the introduction of ¹⁸F can be produced in the form of ¹⁸F-fluorine gas, K¹⁸F and tetramethylammonium ¹⁸F-fluoride. Still other reagents include [¹⁸F]XeF₂, [¹⁸F]AcOF, [¹⁸F]HF, RCH₂CH₂¹⁸F, X—C₆H₄—¹⁸F, ¹⁸F—(CH₂)ₙ—X, and the like (see, PRINCIPLES OF NUCLEAR MEDICINE, 2^{ND} ED. pp. 178-194, (1995)). Fluorine is the smallest replacement for hydrogen and can thus often be introduced to biologically active molecules in place of hydrogen with minimal effect on the structure of the compound. However, it has substantially different electronic character to hydrogen which will often affect the biological activity of the compound. Introduction of a fluorine can also modulate the metabolism of a compound. For these reasons introduction of fluorine is often used as a strategy in drug optimization. See for example, “Fluorine in Bioorganic Chemistry” J. T. Welch and S. Eswarakrishnan, John Wiley and Sons, New York, 1991. A number of pharmaceutical agents contain fluorine and it is known to provide advantages such as reducing metabolism in a parent compound. In addition, facilities more remote from a cyclotron, up to about a 200 mile radius, can make use of ¹⁸F-labeled compounds. Disadvantages of ¹⁸F are the relative scarcity of fluorinated analogs that have functional equivalence to naturally-occurring biological materials, and the difficulty of designing methods of synthesis that efficiently utilize the starting material generated in the cyclotron. Such starting material can be either fluoride ion or fluorine gas. In the latter case only one fluorine atom of the bimolecular gas is actually a radionuclide, so the gas is designated 18F—F. Reactions using ¹⁸F—F as starting material therefore yield products having at most one half the specific radioactivity of reactions utilizing K¹⁸F as starting material. On the other hand, ¹⁸F can be prepared in curie quantities as fluoride ion for incorporation into a radiopharmaceutical compound in high specific activity, theoretically 1.7 Ci/nmol using carrier-free nucleophilic substitution reactions.

Nitrogen-13 (¹³N) is yet another useful positron-emitting radionuclide with a half-life of about 10 minutes and a maximum beta energy (Eₘₐₓ) of 1.2 MeV. ¹³N-ammonia is readily available and has been used to prepare a-number of substituted amines.

The final or penultimate step used in preparing the PET-ready libraries of the present invention uses a cold version of a known PET reagent, a plurality of PET-ready reagents, or a mixture of PET-ready reagents (e.g., methyl iodide, methyl triflate, potassium fluoride, fluorine gas, tetramethylammonium fluoride, tetrabutylammonium fluoride, phosgene, fluoroiodomethane, carbon monoxide, bromofluoromethane, fluoromethyl tosylate, 2-fluoroethyl bromide, 2-fluoroethyl iodide, 2-fluoroethyl tosylate, 2-fluoroethyl triflate, and the like). Alternatively, the final of penultimate step in preparing a PET-ready library can be a step using a reagent for which a PET-labeled substitute is available (see Scheme 4 and corresponding discussion).

In one group of embodiments, the final or penultimate step is one in which each member of the nascent library is treated with the same PET-ready reagent (e.g., methyl iodide, acetyl chloride, potassium fluoride, and the like) to produce a PET-ready library of the invention. In another group of embodiments, the final or penultimate step is one in which each member of the library is treated with a PET-ready reagent selected from a group of PET-ready reagents. In still another embodiment, the final or penultimate step is one in which the nascent library is treated with a plurality of two or more PET-ready reagents.

One of skill in the art would readily understand that the present invention can be used to provide libraries of compounds that readily be labeled for SPECT or planar scintigraphy imaging studies (SPECT-ready libraries). Particularly preferred SPECT labels include ¹²³I and ¹³¹I. ¹³¹Iodine-labeled compounds can also be used for radiotherapy.

Likewise, one of skill in the art would understand that the present invention provides for libraries of compounds that can be readily labeled for autoradiography (autoradiography-ready libraries). Particularly preferred labels include ³H, ¹⁴C, ³²P, and ¹²⁵I.

Methods of Preparing PET-ready Libraries

In another aspect, the present invention provides methods of preparing a positron emission tomography (PET)-ready library of candidate pharmaceutical agents. In general, the methods comprise:

(a) providing a library of compounds; and

(b) treating the library of compounds with a PET-ready reagent or a plurality of PET-ready reagents to produce a PET-ready library of candidate pharmaceutical agents in which each member of the library has been exposed to and preferably has reacted with a PET-ready reagent.

The library of compounds used in this aspect of the invention can be essentially any combinatorial library of compounds wherein each member has a functional group or reactive center that can react with a PET-ready reagent to produce a PET-ready version of a candidate pharmaceutical agent. Suitable functional groups or reactive centers can include a hydroxyl group, an amino group, an aromatic or heteroaromatic ring, an ester or carboxylic acid, a thiol, an aldehyde, an alkyl halide (or other suitable leaving group attached to an alkyl group), a phosphorus-containing group (e.g., a phosphate, phosphonate, phosphinate or phosphine group), a sulfate, a double bond, a triple bond, a strained ring (e.g., an epoxide), or a ketone. A number of reviews are available to guide the practitioner in considering and selecting methods useful for preparing the initial libraries that will be converted to PET-ready libraries. See, for example, Gordon, et al., J. Med. Chem. 37(10):1385-1401 (1994).

In one group of embodiments, the PET-ready library is a solution-based library of compounds. Solution-phase methodologies, can be conducted entirely in the solution-phase or, alternatively, can take advantage of supported reagents which can be easily filtered away from the desired reaction products. A large array of supported reagents are known to those of skill in the art. See, for example, Brummer, et al., Curr. Opin. Drug Discovery Dev. 3(4):462-473 (2000); Thompson, Curr. Opin. Chem. Biol. 4(3):324-337 (2000); and Bhattacharyya, Comb. Chem. High Throughput Screening, 3(2):65-92 (2000). Useful reagents include, for example, supported acids and bases, supported catalysts, supported protecting groups, etc.

In another group of embodiments, the library of compounds is one which is a-solid-phase library (e.g., compounds which are attached to a single or multiple supports). Preferably, the PET-ready library is also prepared on a solid support (e.g., a resin, a glass slide or a bead) using any of a variety of solid-phase synthetic techniques known to those of skill in the art. Alternatively, the library of compounds can be cleaved from the support or supports and treated in solution with a PET-ready reagent. In this embodiment of the invention, the solid supports can be any of those which are known in the art, and may be biological, nonbiological, organic, inorganic, or a combination of any of these. The solid supports can exist as particles, strands, precipitates, gels, sheets, tubing, spheres, containers, capillaries, pads, slices, films, plates, slides, etc. For example, the solid support may be flat, or contain raised or depressed regions on which synthesis takes place. In some embodiments, the solid support will be chosen to provide appropriate light-absorbing characteristics. For example, the support may be a polymerized Langmuir Blodgett film, functionalized glass, Si, Ge, GaAs, GaP, SiO₂, SiN₄, modified silicon, or any one of a variety of gels or polymers such as (poly)tetrafluoroethylene, (poly)vinylidendifluoride, polystyrene, polycarbonate, or combinations thereof. Preferably, the surface of the solid support will contain reactive groups, which could be carboxyl, amino, hydroxyl, thiol, or the like.

Solid-phase synthesis techniques commonly used in peptide or oligonucleotide synthesis can be used, or adapted for use, in the methods of the present invention. Preferably, the solid support is a resin such as, for example, Argogel® or Argopore® (from Argonaut Technologies, Foster City, Calif., USA) or TentaGel® (from Rapp Polymere, Tubingen, FRG). Additionally, devices such as the MicroKANS® from Irori (see www.irori.com) and the “crowns” or “lanterns” from Chiron Technologies (see www.chirontechnologies.com.au) are useful in preparing the present libraries.

In a preferred embodiment, a solid-phase synthesis is performed which meets the following criteria. The compounds are simultaneously synthesized in a parallel synthesis format which is compatible with the standard techniques of organic synthesis. The final compounds are produced individually (not as mixtures). The quantity of compound generated is greater than 1 mg and the compound should be generated in sufficiently pure form to allow for its direct testing. In another preferred embodiment, sample handling is carried out using automated systems for speed, accuracy and precision. In yet another preferred embodiment, the library members are readily separable from by-products and reagents.

Still other solid phase methods are those which utilize beads as the solid support, and produce “bead-based libraries.” See, for example, WO 96/00391, U.S. Pat. No. 5,639,603 and U.S. Pat. No. 5,708,153.

In yet another group of embodiments, the PET-ready library is a library in which each member is “tagged” for identification. In this embodiment of the invention, libraries can be prepared and tagged as described in, for example, U.S. Pat. Nos. 5,789,162 and 5,708,153. See also, Maclean, et al., Proc. Natl. Acad. Sci, USA 94:2805-2810 (1997).

Devices for the preparation of combinatorial libraries are commercially available (see, e.g., “Benchmark Vantage” series: Advanced Chem Tech, Louisville Ky., (www.peptide.com) “Trident” or “Quest” or “Nautilus” synthesizers, Argonaut Technologies, Foster City, Calif. (www.argotech.com).

In addition to providing a route for the facile addition of positron emitting nuclei that can be detected by PET, another aspect of the invention provides a means of adding gamma-emitting, beta-emitting, or alpha-emitting nuclei that can be detected by SPECT, autoradiography or other means.

Hence, in another aspect of the invention, a method is provided for determining the distribution of an active agent in a tissue using a SPECT-ready library. This method comprises (a) screening a SPECT-ready library of potential agents against a biological target; (b) identifying at least one the potential agents as an active agent; (c) preparing a SPECT-labeled version of the active agents, wherein the SPECT-label is incorporated in the final or penultimate step of active agent synthesis; (d) administering the SPECT-labeled version to a subject; and (e) measuring the distribution of the active agent. In a preferred embodiment, the SPECT-label is chosen from ¹²³I or ¹³¹I.

Similarly, a method is provided for determining the distribution of an active agent in a tissue using an autoradiography-ready library. This method comprises (a) screening an autoradiography-ready library of potential agents against a biological target; (b) identifying at least one the potential agents as an active agent; (c) preparing an autoradiography-labeled version of the active agents, wherein the autoradiography-label is incorporated in the final or penultimate step of active agent synthesis; (d) administering the autoradiography-labeled version to a subject; and (e) measuring the distribution of the active agent. In a preferred embodiment, the autoradiography-label is chosen from ³H, ¹⁴C, ³²P or ¹²⁵I.

The invention is illustrated below with examples in which (i) an existing support-bound library is converted to a PET-ready library, and (ii) a PET-ready library is prepared then cleaved from the support.

Modification of an Existing Library

In one embodiment, a support-bound library is prepared in which there are multiple sites of diversity. The library is then cleaved from the solid support and treated with a single PET-ready reagent to form a PET-ready library.

Illustrating this aspect of the invention is the preparation of PET-ready libraries of ortho-fluorophenols in Scheme 1a. In this Scheme, a substituted phenolic ketopiperazine is attached to a solid support (i) as described in Zhu, et al., Tetrahedron Lett. 39:7479-7482 (1998). The PG group represents a protecting group for the piperazine nitrogen atom. The R₁ group provides a first site of diversity in generating the library and can be any group that is compatible with the subsequent diversity-generating reactions (e.g., alkyl, alkoxy, heterocyclic moieties, etc.). The tethered ketopiperazine (i) can then be alkylated with an arylalkylhalide (e.g., benzyl bromide) which can provide a second site of diversity, to produce a family of tethered substituted ketopiperazines (ii). Additional diversity is generated by deprotecting the piperazine nitrogen and acylating the newly produced amine with, for example, a suitable carboxylic acid, acid chloride, carboxylic anhydride and the like (R³—CO₂H, R³—COCl, and (R³—CO)₂—O) to produce the library (iii). Thus, an initial library is prepared having three sites of diversity that depend only on the availability of substituted (hydroxy)benzyl halides (see, ibid.), arylalkyl halides, and carboxylic acids (or their reactive derivatives). The library can then be removed from the solid support (typically a resin or bead) and fluorinated using fluorine gas as a PET-ready reagent to provide a library of ortho-fluorophenols (iv) as shown. In this manner, the PET-ready reagent does not create any additional diversity in the library but provides a functional group (in this case a fluorine atom) that is ready for labeling. Upon identifying an active agent in the library, the synthetic methods are in place to introduce a ¹⁸F-label, using ¹⁸F-fluorine gas in the final step of synthesis.

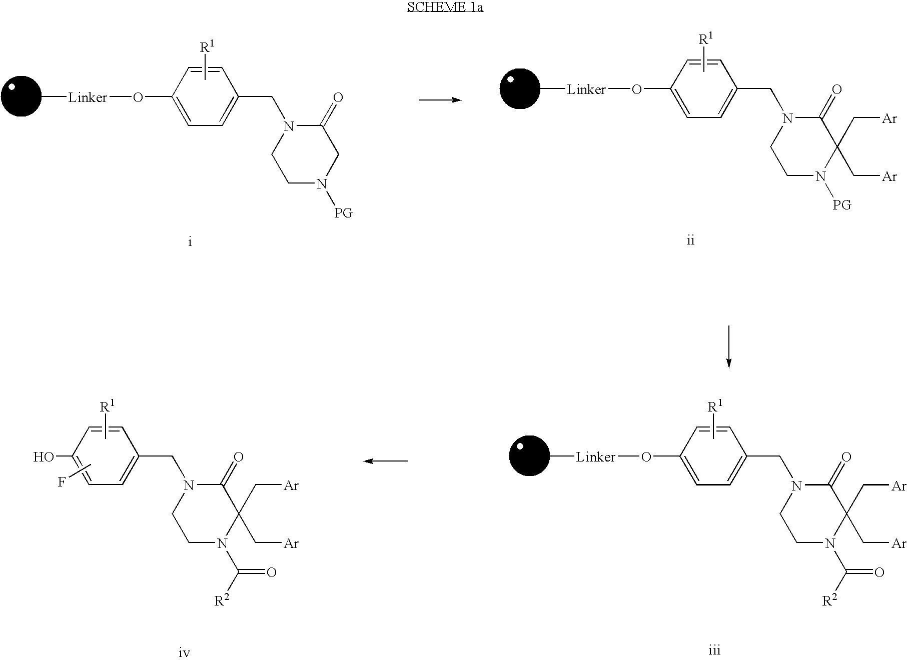

Alternatively, the library of phenols (v) prepared as described in Zhu, et al., Tetrahedron Lett. 39:7479-7482 (1998) can be treated with a plurality of PET-ready reagents to create an additional site of diversity. In this embodiment, the phenols can be treated with, for example, methyl iodide, 2-bromo-1-fluoroethane and other PET-ready alkyl iodides (see Scheme 1b) to provide vi, vii and viii. The atoms which can be labeled in subsequent syntheses are shown with (*).

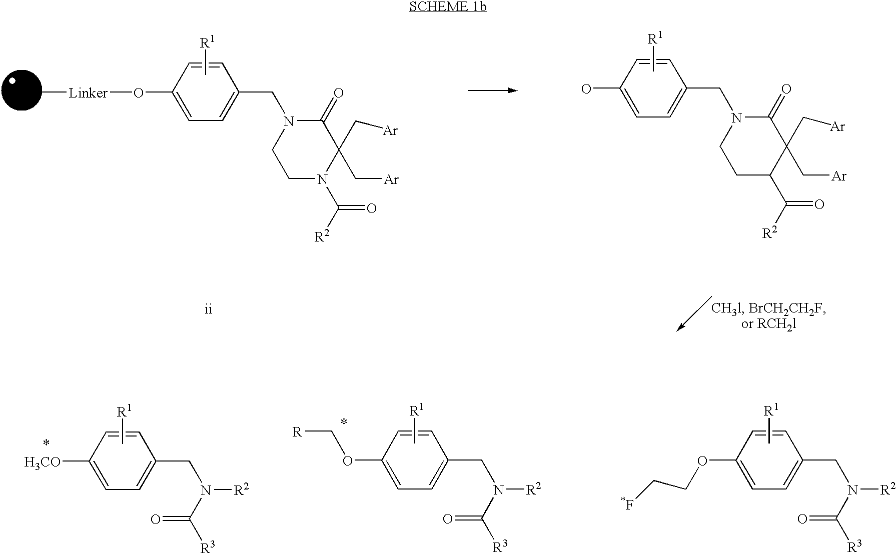

Similarly, the substituted thiazolidinone pharmacophore (x) shown in Scheme 1c can be treated with methyl iodide as above to provide (xi), and then cleaved from the support to provide another library (xii) which is PET-ready (using ¹¹C—CH₃I in place of “cold” methyl iodide when a labeled compound is desired). See, Holmes, et al. J. Org. Chem. 60(22):7328-33 (1995).

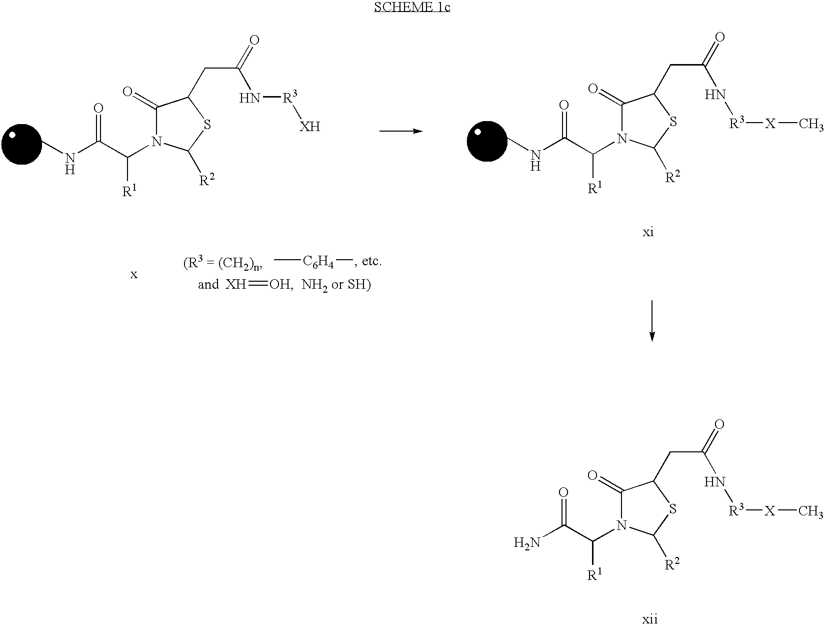

Alternatively, the reactive group —XH can be treated with a plurality of PET-ready reagents, as described for Scheme 1b, to create additional diversity in the library. In other embodiments, libraries can be prepared that are linked to the support via an ester group (rather than the amide which is shown). Cleavage from the support provides a library of carboxylic acids that can be converted, with the appropriate reagents, to methyl esters or fluoroalkyl esters.

De novo Library Synthesis

One example of de novo synthesis of a PET-ready library can be illustrated using, for example, a vapor-phase Hoffman elimination to generate tertiary amines (Scheme 2, see, Brown, J. Comb. Chem. 1:283-285 (1999)). As shown in Scheme 2, a resin having a suitable Michael acceptor (xiii) can be treated with a secondary amine to provide (xiv) which can be treated with, for example, methyl iodide, 1-bromo-2-fluoroethane (“cold” versions of the PET reagents ¹¹C—CH₃I and ¹⁸F—CH₂CH₂—Br, respectively) or another PET-ready alkylating agent to provide a support-bound library of quaternary ammonium groups (xv). Treatment of the nascent library xv with a suitable base (e.g., ammonia in the vapor phase) results in release of the tertiary amines thus produced to provide the library xvi. This route is particularly useful as a variety of secondary amines are commercially available and can be attached to a resin such as, for example, xiii. Primary amines can also be used (R²=H) and modification of groups R² and/or R¹ after attachment of the amine to the resin will allow generation of even larger numbers of compounds.

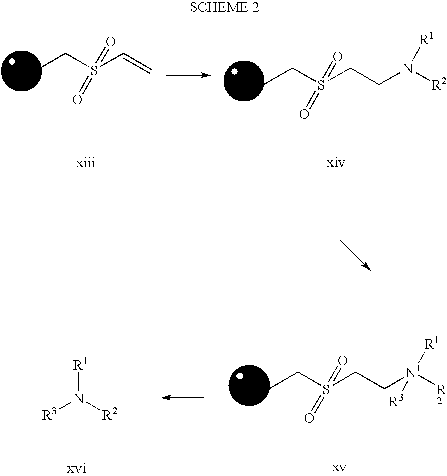

Another example of de novo synthesis of a PET-ready library can be illustrated using, for example, the synthesis of 2-aminopyrimidines shown in Scheme 3. As shown in this scheme, a resin having an attached 1,3-dicarbonyl group (xvii) is treated with an aldehyde and 2-methyl-2-thiopseudourea to form a support-bound heterocyclic scaffold (xviii) which is then oxidized to a pyrimidine moiety having a methylsulfonyl leaving group in the 2-position of the pyrimidine ring (xix). Displacement of the leaving group with a PET-ready reagent (shown here as R³—NH₂) produces a PET-ready library of pyrimidine derivatives (xx).

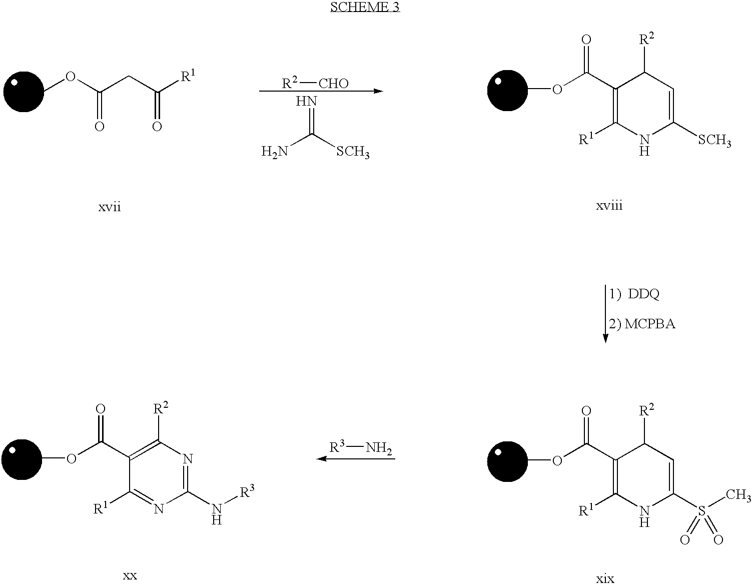

In this scheme, R³—NH₂, can then be ¹¹C-methylamine, or any alkylamine available in, for example, ¹³N- or ¹¹C-labeled form. Alternatively, R³—NH₂ can be replaced with HO—R³—NH₂, H₂N—R³—NH₂, secondary diamines (e.g., piperazine, N,N′-dimethylethylenediamine), or even unsymmetrical diamines in protected or unprotected form. The use of these reagents then provides an additional site that can be derivatized by, for example, alkylation using reagents such as methyl iodide, fluoroethyl bromide and the like as described above.

In still other embodiments, the methods of preparing PET-ready libraries is meant to include those reactions wherein the final or penultimate step does not use a PET-ready reagent, but the process can be carried out with PET-labeled reagents in an alternative path to produce a labeled compound. For example, in Scheme 4 below, the first two processes use PET-ready reagents (phosgene and carbon monoxide are both available in ¹¹C-labeled form) while the third process uses a substituted carbamoyl chloride to arrive at the same urea derivative. Accordingly, the third process is also considered a PET-ready process even though the substituted carbamoyl chloride is not readily available in labeled form.

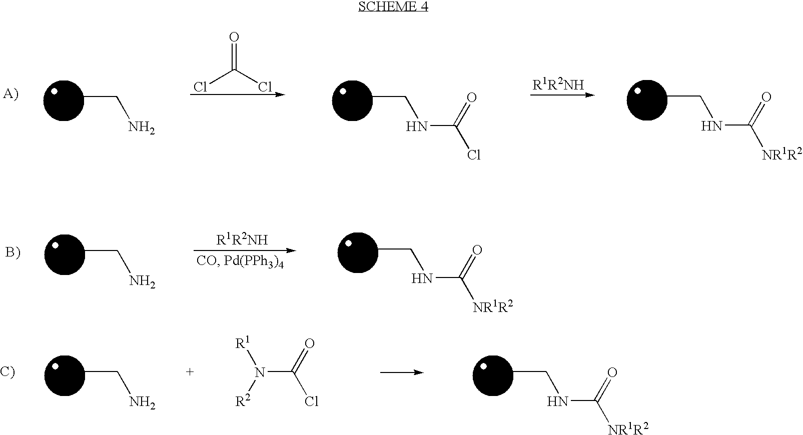

Methods of Using PET-ready Libraries

In yet another aspect, the present invention provides a method for determining the distribution of an active agent in a tissue, comprising:

(a) screening a PET-ready-library of candidate pharmaceutical agents against a biological target;

(b) identifying at least one of said candidate pharmaceutical agents as an active agent;

(c) preparing a PET-labeled version of said active agent, wherein the preparing comprises incorporating a PET-label into the final or penultimate step of active agent synthesis;

(d) administering the PET-labeled version of said active agent to a subject; and

(e) measuring the distribution of the active agent in at least one tissue of the subject.

In this aspect of the invention, the PET-ready library can be any of the libraries described above or prepared by the methods described above. Typically, the libraries will have from about 12 to about 100,000 candidate pharmaceutical agents, but may have from 100,000 to 1,000,000 or more. The libraries can be pools of candidate agents or can be available as discrete compounds (e.g., one compound or candidate agent per well of a 96-well, 384-well, 864-well or 1536-well plate). Additionally, the biological target can be essentially any target molecule (e.g., a receptor, enzyme, gene, promoter, etc.) or pathway for which modulation of its action is desired. The biological target can be present in, for example, a solution-based assay or a cell-based assay. Alternatively, the target can be attached to a solid support and the libraries described herein can be screened against the support-bound target.

Identifying an active agent from among the candidate pharmaceutical agents will typically involve selecting one or more compounds that achieve a threshold level of activity (e.g., as an agonist, antagonist, inhibitor, binder, modulator of gene expression, channel blocker, channel opener, and the like). Preferably, the screening and identifying is carried out using a high-throughput screen such as those described in, for example, Gordon et al., J. Med. Chem. 37(10):1385-1401 (1994); or any of U.S. Pat. Nos. 5,902,726, 5,783,398, 5,705,344 and 5,635,349.

Once an active agent is identified, the agent will be prepared in a PET-labeled form. Typically, the synthesis is carried out using the same strategy as that used in the initial preparation (e.g., solid phase or solution phase methods). In this manner, a PET-labeled form of the active agent can be prepared by simply substituting a PET-labeled reagent for the PET-ready reagent which was used in the final or penultimate step of the PET-ready library preparation.

The PET-labeled version of the active agent can then be administered to a subject using essentially any available means for administering a compound. The subject can be human or animal and the administering can be for experimental and/or diagnostic purposes. Typically, an image-generating amount of the active agent, labeled with the appropriate isotope is administered. An image-generating amount is that amount which is at least able to provide an image in a PET scanner (or a SPECT scanner or autoradiography camera in other embodiments of the invention). The amount will also depend on the scanner's detection sensitivity and noise level, the age of the isotope, the body size of the subject and route of administration, all such variables being exemplary of those known and accounted for by calculations and measurements known to those skilled in the art.

Following administration, the distribution of the PET-labeled version of the active agent is measured in at least one tissue of the subject. Measurements will be taken by appropriate scanners, as noted above. In one group of embodiments, the scanner is a MicroPET high-resolution positron emission tomography scanner (see, Cherry, et al., “MicroPET: a High Resolution PET Scanner for Imaging Small Animals”; IEEE Transactions on Nuclear Science, (1997) Vol. 44, No. 3, pp. 1161-1166; and Cherry, et al., in “Quantitative Functional Brain Imaging by PET”; Academic Press, (1998)).

Methods Using PET-ready Linking Groups and other Reagents

While the above disclosures have focused on the preparation of PET-ready libraries, other features of the present invention are applicable in the context of libraries or in the synthesis of discrete compounds. More particularly, linker strategies and reagents are now described that can be applied to the solid phase synthesis of PET-ready libraries or to solid phase synthesis of PET-labeled ligands (e.g., radiopharmaceuticals labeled with positron-emitting isotopes).

In this aspect, the invention provides a method for preparing a PET-labeled compound, the method comprising:

(a) providing a precursor compound covalently attached to a solid support;

(b) contacting said precursor compound with a PET-labeled reagent to produce a composition comprising a PET-labeled compound portion attached to said solid support by a linking group; and

(c) selectively removing said PET-labeled compound from said composition.

In one group of embodiments, the PET-labeled compound is selectively removed from the support under conditions whereby any unreacted precursor compound remains covalently attached to said solid support.

In a related aspect, the PET-labeled compound is removed from the solid support at a rate which is faster than the unreacted starting compound or other side products. Preferably, the PET-labeled compound is removed at a rate at least 30%, more preferably at least 40% and still more preferably at least 50% more rapidly than unreacted starting materials.

One of skill in the art will further appreciate that the above method can be practiced with a variety of precursor compounds, solid supports and linking groups that allow for the selective removal of the final product (a PET-labeled compound) from the support while leaving substantially all of the unreacted precursor compounds attached to the support. More particularly, one of skill in the art will appreciate that safety-catch linkers are particularly useful for this aspect of the invention.

A variety of safety catch linkers are known in the art and are useful in the present invention (see, for example, Kenner, et al., Chem. Commun. 1971, 636-637; James, Tetrahedron 1999, 55, 4855-4946; Backes, et al., Curr. Opinion Chem. Biol. 1997, 1, 86-93; Backes, et al., J. Am. Chem. Soc. 1994, 116, 11171-11172; Backes, et al., J. Am. Chem. Soc. 1996, 118, 3055-3056; Backes, et al., J. Org. Chem. 1999, 64, 2322-2330; Backes, et al., J. Org. Chem. 1999, 64, 5472-5478; Link, et al., Tetrahedron Letts. 1998, 39, 5175-5176; Routledge, et al., Tetrahedron Lett. 1997, 38, 1227-1230, Lee, et al., J. Org. Chem. 1999, 64, 3454-3460; Wade, et al., J. Comb. Chem. 2000, 2, 266-275; Hulme, et al., Tetrahedron Letts. 1998, 39, 7227-7230; Panke, et al., Tetrahedron Letts. 1998, 39, 17-18; and Nicolaou, et al., Angew. Chem. Int. Ed. 2000, 39, 1084-1088). Briefly, safety catch linkers are those groups that typically require a two-step pathway for release of a particular agent that is prepared on the linker. Unreacted starting materials or incompetent products remain attached to the solid support. Exemplary of such linkers is a REM linker depicted in Scheme 5.

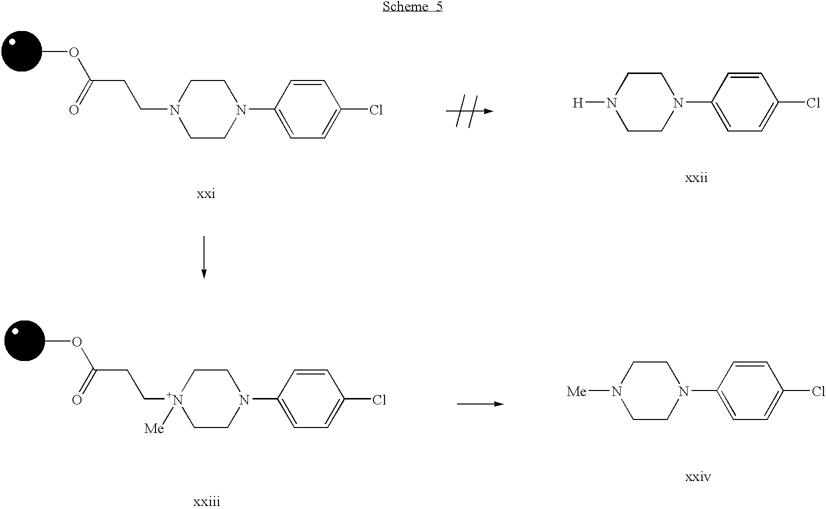

In this scheme, cleavage of a resin-bound amine xxi (a substituted piperazine) is achieved only following reaction with an alkylating agent such as methyl iodide. Thus, treatment of xxi with methyl iodide produces xxiii which can be released from the resin to provide the target xxiv, while the unreacted starting compound xxii is not released. In this manner, the target is provided which is relatively free of side products and parent compounds. This safety catch strategy is particularly well suited for PET labeling as other methods require a time-consuming separation of starting materials and product—a process that can significantly reduce radiochemical yields due to isotope decay. Additionally, (with reference to Scheme 5, above) when using a safety catch linker or resin, an excess of the resin-bound material can be treated with traces of ¹¹C-methyl iodide to promote efficient use of the expensive reagent. Following incubation of the resin and reagent for an appropriate time, any unreacted reagent is simply washed off the resin. Subsequent treatment with base will cleave the ¹¹C-methylated material to provide the desired product while leaving unreacted materials attached to the resin. One of skill in the art will further appreciate that materials left attached to the resin are not lost, but can be subjected to the reaction conditions by treatment with a second aliquot of PET-labeled reagent.

Other advantages of this process include facile automation as solutions can be easily delivered to the resin, resulting in both operator safety and convenience.

In addition to the REM linker illustrated in Scheme 5, the present invention can be practiced with other safety catch linkers such as a sulfone-REM linker (see, Kroll, et al., Tetrahedron Letts., 1997, 38, 8573-8576). In a particularly preferred embodiment, the linker is a “reversed Kenner” linker (depicted in Scheme 6 for the preparation of substituted sulfonamides).

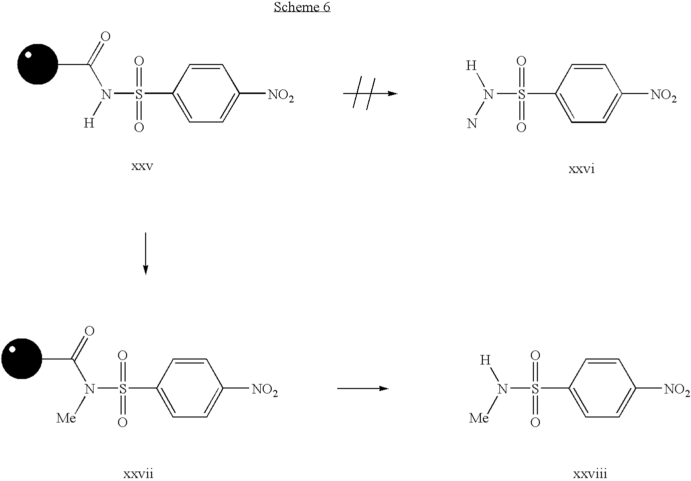

The reversed Kenner strategy illustrated in Scheme 6 relies on principles similar to those of the REM linkers. In particular, the strategy uses an alkylation step (to produce xxvii) which renders the linkage sensitive to basic conditions for cleavage. As in the REM process of Scheme 5, the reversed Kenner strategy does not allow unreacted starting material xxvi to be released from the resin. Once again, methyl iodide provides a convenient reagent for the alkylation step, but other suitable reagents are also available in PET-labeled form.

Scheme 7 illustrates yet another type of safety catch strategy which is amenable to processes for preparing PET-labeled compounds.

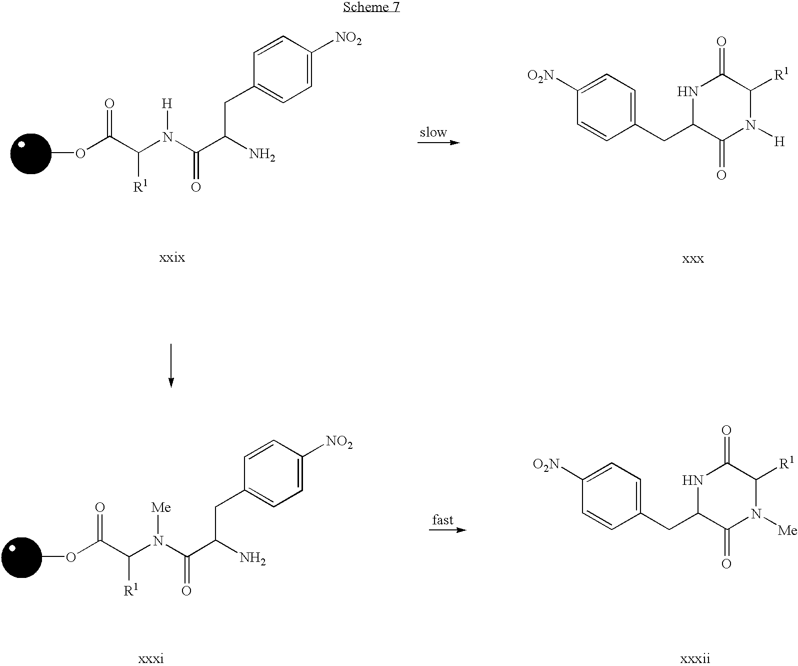

In this method, the safety catch exploits the relative rates of subsequent processes, following introduction of a PET-reagent. For example, the cyclization of dipeptides (internal N-alkyl amides versus unsubstituted amides) provides an avenue for the release of N-alkyl product xxxii at a much faster rate than the N—H product xxx. While dissimilar from the other methods in releasing unreacted product from the linker, enrichment factors of 40%, more preferably 60% and still more preferably 80% or more can be achieved. Scheme 8 illustrates a practical route for the preparation of PET-labeled diketopiperazines.

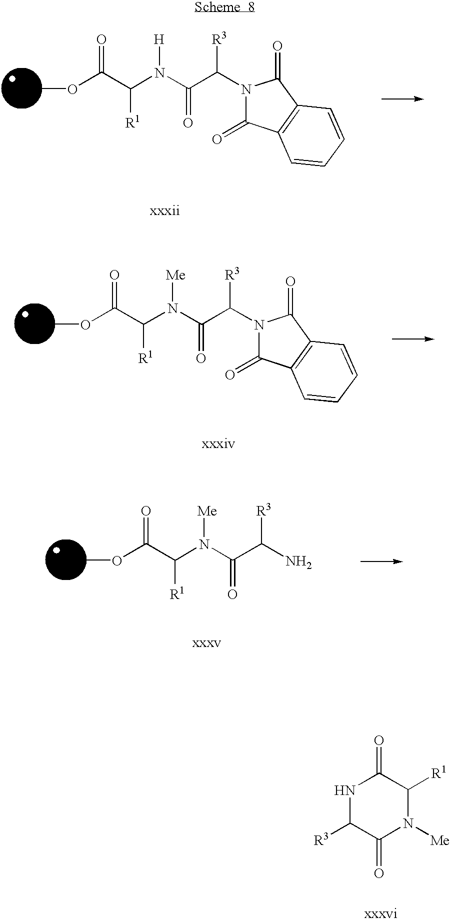

In this scheme, a phthalimido-protected dipeptide xxxiii is treated with a PET-labeled (or PET-ready) reagent, for example, methyl iodide, to produce the N-alkylated dipeptide xxxiv. Removal of the protecting group from the N-terminus provides a dipeptide xxxv that undergoes an internal cyclization and concommitant release from the solid support to provide the diketopiperazine xxxvi.

The Reverse Kenner Safety Catch Linker

In another aspect the present invention provides support bound safety-catch linker having the formula:

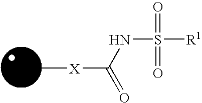

wherein the shaded sphere represents a solid support; X represents a substituted or unsubstituted (C₁-C₂₀)alkylene; and R¹ represents a substituted or unsubstituted aryl, substituted or unsubstituted heteroaryl, substituted or unsubstituted (C₁-C₂₀)alkyl, substituted or unsubstituted aryl(C₁-C₈)alkyl, or a substituted or unsubstituted heteroaryl(C₁-C₈)alkyl.

In preferred embodiments, X is a (C₁-C₈)alkylene group which is tethered to the solid support via, for example, an ether, amide, ester, siloxane or amine linkage, or a combination thereof.

The above composition is particularly useful in the preparation of substituted aryl sulfonamides. Accordingly, in preferred embodiments, R¹ is a substituted or unsubstituted aryl group. More preferably, R¹ is a substituted or unsubstituted phenyl or naphthyl group.

In still further preferred embodiments, X is an unsubstituted (C₁-C₈)alkylene group and R¹ is a substituted or unsubstituted aryl group.

One of skill in the art will appreciate that the “reverse” Kenner safety catch linker is one that has utility in PET-ready, PET-labeled, and unlabeled synthetic methods.

The following examples are offered to illustrate, but not to limit the claimed invention.

### EXAMPLES

Reagents and solvents used below can be obtained from commercial sources such as Aldrich Chemical Co. (Milwaukee, Wis., USA). ¹H—NMR spectra were recorded on a Varian 400 MHz NMR spectrometer. Significant peaks are tabulated in the order: number of protons, multiplicity (s, singlet; d, doublet; t, triplet; q, quartet; m, multiplet; br s, broad singlet) and coupling constant(s) in Hertz. Electron Ionization (EI) mass spectra were recorded on a Hewlett-Packard mass spectrometer. Mass spectrometry results are reported as the ratio of mass over charge, followed by the relative abundance of each ion (in parentheses).

Abbreviations used in the Examples below have the accepted meanings known by those of skill in the art. For example: NMP, N-methyl pyrrolidine; TFA, trifluoroacetic acid; DCM, dichloromethane; DIEA, diisopropylethylamine; FMOC, fluorenylmethoxycarbonyl; DMAP, 4-dimethylaminopyridine, mL, milliliter; mg, milligram; μL, microliter; h, hour; min, minutes.

### Example 1

This example illustrates the REM safety catch linker approach to the preparation of PET-ready compounds.

Preparation of Wang Acrylate Resin

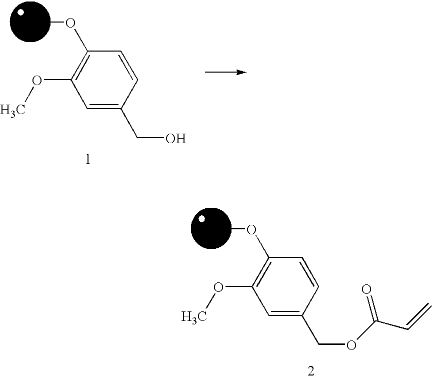

ArgoGel-Wang resin 1 (San Carlos, Calif.; 160 g, 0.39 mmol/g; 62.4 mmol) was stirred gently in anhydrous dichloromethane (DCM; 1.5 L) and diisopropyl-ethylamine (120 mL, 673 mmol, 10.8 eq) added, followed by dimethylaminopyridine (0.5 g; 4.1 mmol, 0.066 eq). The vessel was flushed with nitrogen gas and acryloyl chloride (52 mL, 624 mmol, 10 eq) was added dropwise with stirring over 30 min such that the reaction temperature did not exceed 30° C. Stirring under nitrogen was continued for 3 h at which time the mixture was filtered and the resin was washed with DCM (5×500 mL), N-methylpyrrolidine (NMP; 3×500 mL), DCM (3×500 mL), and methanol (5×500 mL) before drying overnight under vacuum at 40° C. to give resin 2 as a cream-colored solid (163.75 g). Completion of the reaction was confirmed by the disappearance of the benzylic protons of the starting resin as judged by solid-phase magic-angle spinning (SP-MAS) NMR.

Michael Addition of Secondary Amine

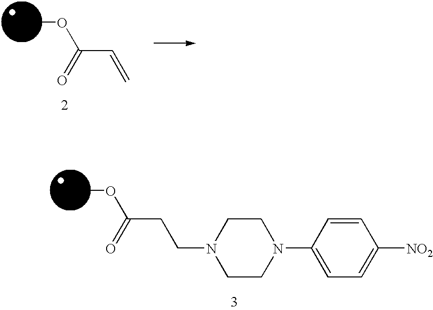

Acrylate resin 2 (8.0 g, 0.39 mmol/g, 2.4 mmol) was treated with a solution of N-(4-nitrophenyl)-piperazine (4.97 g, 24 mmol, 10 eq) in dimethylformamide (DMF; 50 mL) and shaken at 20° C. for 18 h. The mixture was filtered and the resin washed and dried as described above (40 mL portions of wash solvents) to give resin 3 as a pale yellow solid. Completion of reaction was confirmed by disappearance of the signals for the acrylate protons of resin 2 in SP-MAS NMR.

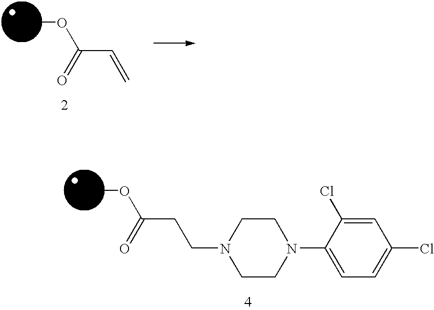

Acrylate resin 2 (8.0 g, 0.39 mmol/g, 2.4 mmol) was treated with a solution of N-(2,4-dichlorophenyl)-piperazine (4.97 g, 24 mmol, 10 eq) in dimethylformamide (DMF; 50 mL) and shaken at 20° C. for 18 h. The mixture was filtered and the resin was washed and dried as described above for resin 2 (40 mL portions of wash solvents) to give resin 4 as a pale yellow solid. Completion of reaction was confirmed by disappearance of the signals for the acrylate protons of resin 2 in SP-MAS NMR.

Methylation of Secondary Amine

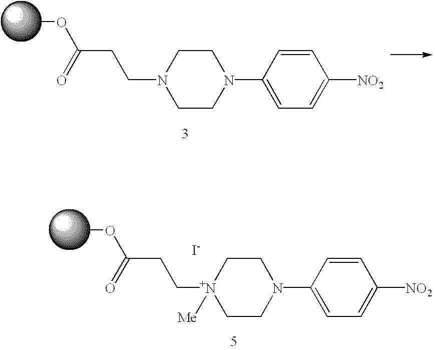

N-(4-Nitrophenyl)-piperazine resin 3 (6.0 g, 0.39 mmol/g, 2.34 mmol) was treated with methyl iodide (1.46 mL, 23.4 mmol, 10 eq) and DMF (640 mL) then shaken at 20° C. for 18 h. The resin was washed and dried to give resin 5.

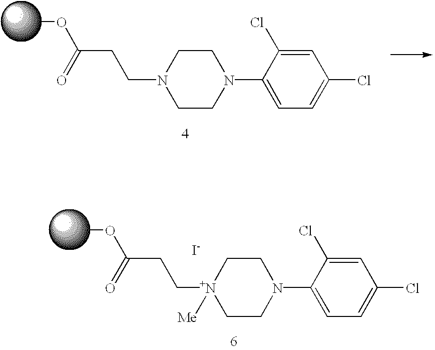

N-(2,4-dichlorophenyl)-piperazine resin 4 (10.0 g, 0.39 mmol/g, 3.9 mmol) was treated with methyl iodide (2.6 mL, 39 mmol, 10 eq) and DMF (60 mL) then shaken at 20° C. for 18 h. The resin was washed and dried to give resin 6.

Release of N-Methyl Tertiary Amine

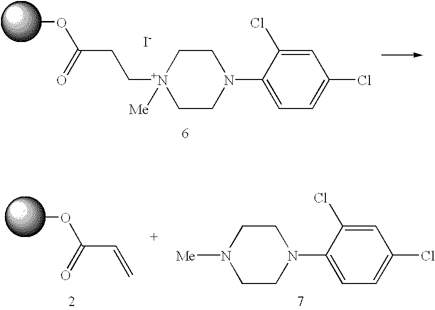

Method 1

Methylated resin 6 (0.5 g, 0.3 mmol/g, 0.15 mmol) was treated with 2M ammonia in methanol (0.975 mL, 1.95 mmol, 13 eq) and DCM (10 mL). The mixture was shaken at 20° C. for 10 min then filtered and the resin washed with DCM (3×10 mL). The combined filtrate and washings were evaporated under reduced pressure to give crude product 7 as a yellow solid (0.027 g, 82%) and the structure confirmed by mass spectrometry and NMR. SP-MAS NMR of the resin was consistent with structure 2.

Method 2

Methylated resin 6 (0.5 g, 0.3 mmol/g, 0.15 mmol) was treated with DIEA (0.34 mL, 1.95 mmol, 13 eq) and acetonitrile (6 mL). The mixture was heated to 50° C. for 10 min then drained and the resin was washed with DCM (3×10 mL). The combined filtrate and washings were evaporated under reduced pressure to give crude product 6 as a yellow solid which was dissolved in ethyl acetate (1 mL) and passed through a column containing a mixture of powdered silica and potassium carbonate (0.5 g each). The column was washed with ethyl acetate and the washings evaporated under reduced pressure to give product 7 as a yellow solid (0.029 g, 88%). The structure was confirmed by mass spectrometry and NMR. SP-MAS NMR of the resin was consistent with structure 2.

Kinetics of Methylation

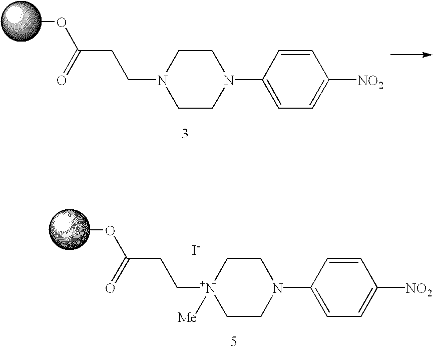

4-Nitrophenylpiperazine resin 3 (0.5 g, 0.195 mmol) was treated with methyl iodide (0.060 mL, 0.975 mmol, 5 eq) and anhydrous DMF (6 mL). The mixture was heated to 50° C. for 30 min and the resin was filtered and washed with DMF (3×10 mL) and DCM (5×10 mL), then cleaved using method 2 (above) to give the desired product 5 (0.027 g, 82%) as determined by MS and NMR.

Repetitive Cleavage

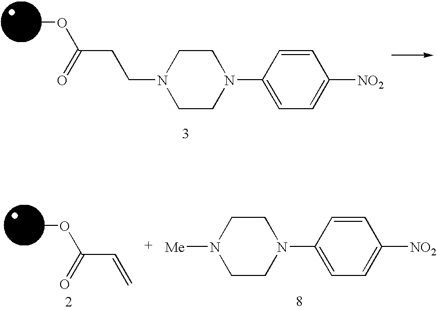

4-Nitrophenylpiperazine resin 3 (1.0 g, 0.39 mmol/g, 0.39 mmol) was treated with methyl iodide (5 μL, 0.08 mmol, 0.2 eq) and heated to 50° C. for 30 min then drained and the resin was washed with DMF and DCM. The resin was then treated with 2M ammonia in methanol (4 mL) and shaken for 20 min at 20° C. The mixture was filtered and the resin washed with DCM (10 mL×3). Combined filtrates were evaporated to give N-(4-nitrophenyl)-N′-methylpiperazine 8 (4.0 mg, 30% of theoretical). The resin was further washed with DMF (10 mL×3) and subjected to further methylation/cleavage as above to give a second portion of the desired material 8 (4 mg, 30%). A third repeat gave another portion of product 8 (7 mg, 52%).

### Example 2

This example illustrates the preparation of PET-ready compounds using a reverse Kenner linker approach.

Preparation of Resin and General Approach

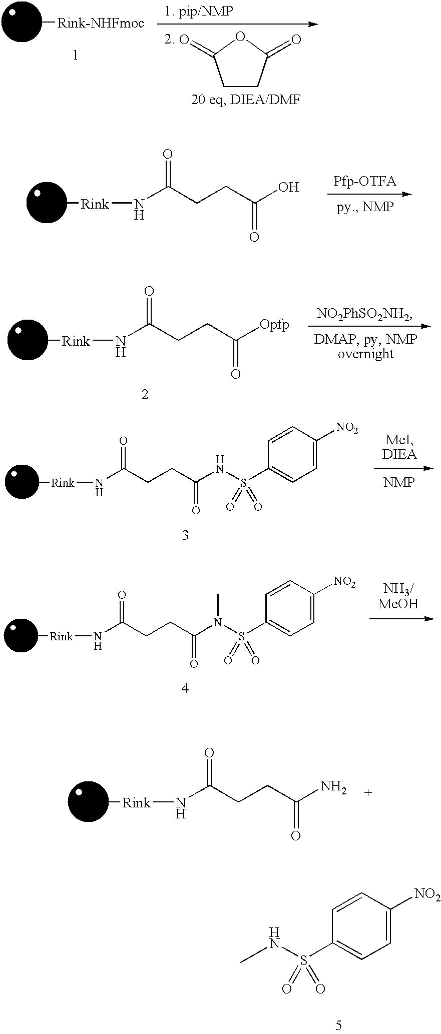

ArgoGel-Rink-NH-Fmoc resin (1, 500 mg, 0.36 mmole/g, 0.18 mmole) was treated with 2 mL of 20% (v/v) piperidine/N-methylpyrrolidine (NMP) solution at room temperature for 20 min before being drained and washed with NMP (5×5 mL). To the resin was added a mixture of succinic anhydride (360 mg, 3.6 mmole, 20 eq) and DIEA (630 μL, 3.6 mmole, 20 eq) in 2 mL of NMP. After shaking at room temperature for 30 min, the resin was filtered and washed with NMP (7×5 mL). It was further treated with a 1:1:1 mixture of pentafluorophenyl trifluoroacetate, pyridine and NMP (2 mL) at room temperature for 20 min followed by a brief wash with NMP (2×5 mL). To the resulted resin (2) was immediately added a solution of 4-nitrobenzenesulfonamide (364 mg, 1.8 mmole, 10 eq), pyridine (730 μL, 90 mmole, 50 eq) and dimethylaminopyridine (2 mg, 0.018 mmole, 0.1 eq) in 2 mL of NMP. The reaction was allowed to proceed at room temperature for 16 hours before it was drained and washed sequentially with NMP (5×), MeOH (3×), dichloromethane (DCM, 3×) and anhydrous NMP (3×) to afford resin 3. The resin 3 was heated at 80° C. with MeI (112 μL, 1.8 mmole, 10 eq) and DIEA (157 μL, 0.9 mmole, 5 eq) in 2 mL of anhydrous NMP for 10 min and then washed well with NMP, DCM and MeOH. Ammonia (1.0 M in MeOH) treatment of the resin for 5 min followed by filtration and concentration of the filtrate gave 35.3 mg (90.7%) of N-methyl-4-nitrobenzenesulfonamide (5) as off-white crystal.

Preparation of N-methyl-4-chlorobenzenesulfonamide

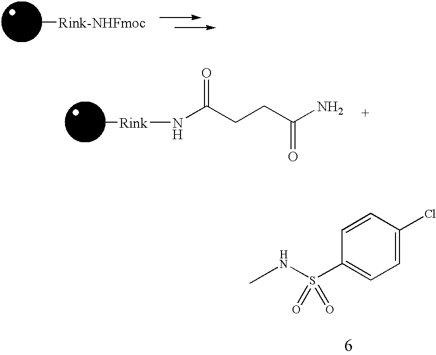

N-methyl-4-chlorobenzenesulfonamide (6) was synthesized from 4-chlorobenzene sulfonamide by means of standard procedure described above and was isolated as off-white crystal with 98.1% yield.

Preparation of N-methyl-benzenesulfonamide

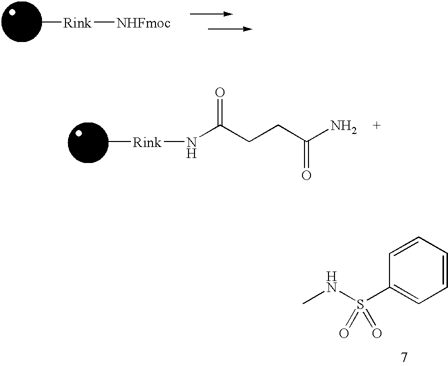

N-methyl-benzenesulfonamide (7) was synthesized from benzylsulfonamide by means of standard procedure described above and was isolated as off-white crystal with 91.8% yield.

Preparation of N-methyl-4-methoxybenzenesulfonamide

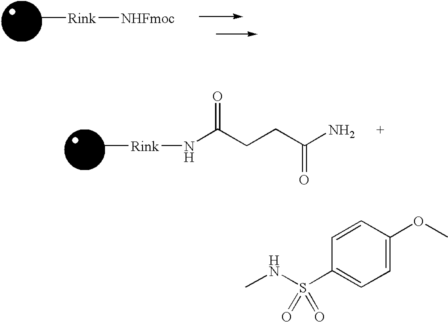

N-methyl-4-methoxybenzenesulfonamide was synthesized from 4-methoxybenzenesulfonamide by means of standard procedure described above and was isolated as off-white solid with 74.2% yield.

Preparation of N-methyl-2-methylbenzenesulfonamide

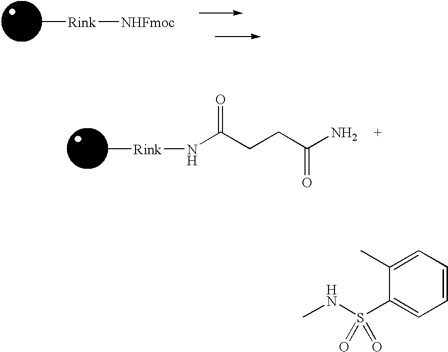

N-methyl-o-toluenesulfonamide was synthesized from o-toluenesulfonamide by means of standard procedure described above and was isolated as off-white solid with 80.9% yield.

### Example 3

This example illustrates the kinetics of methylation using a reverse Kenner linker as described above.

Five portions of resin 3 (700 mg each, 0.25 mmole) were treated with a solution of MeI (157 μL, 2.5 mmole, 10 eq) and DIEA (219 μL, 1.25 mmole, 5 eq) in 2 mL of NMP at room temperature for 10, 30, 90, 270 and 810 min, respectively. The resins were washed with NMP (6×), MeOH (3×) and DCM (3×). A small portion of each resin was cleaved by 50% TFA/DCM for 20 min for analysis. Comparison of UV traces of starting materials and products on LC/MS indicated that methylation was 44% completed at 10 min; 91.9% completed at 30 min; 100% completed after 90 min.

Methylation at 50° C. under similar condition indicated that reaction was 100% completed after 10 min.

### Example 4

This example illustrates the stoichiometry and reusability of reagents when employing a reverse Kenner linker.

Resin 3 (1.0 g, 0.36 mmole) was treated with a solution of MeI (4.5 μL, 0.072 mmole, 0.2 eq) and DIEA (13 μL, 0.072 mmole; 0.2 eq) in 4 mL of NMP at 100° C. for 25 min, followed by washing with NMP (6×), MeOH (3×) and DCM (3×). The above step was repeated three times before a final treatment with 1.0 equivalent of MeI (22.5 μL, 0.36 mmole) and DIEA (65 μL, 0.036 mmole) in 4 mL of NMP under ambient conditions. A small portion of resin from each step was cleaved by 50% TFA/DCM for 20 min for analysis. Comparison of UV traces of starting materials and products on LC/MS indicated that methylation was 25-35% completed compared to theoretical yield for all cases.

### Example 5

This example illustrates the preparation of a PET-ready compound/library using the Ugi reaction. This reaction involves a four-component, one-pot condensation of an aldehyde, a carboxylic acid, an isonitrile and an amine to provide an N-acyl amino acid amide. A number of such reagents are, or may be available as positron-emitting reagents, such as formaldehyde or 4-fluorobenzaldehyde, formic acid, acetic acid, or 4-fluorobenzoic acid, methyl isonitrile or 4-fluorobenzyl isonitrile, methylamine or 4-fluorobenzylamine. In addition, any one of the reactants can be attached to a polymer support. As described below, the reaction can be carried out using an aldehyde as the PET-ready reagent (e.g., formaldehyde is available in ¹¹C labeled form), and having an amine component tethered to the support.

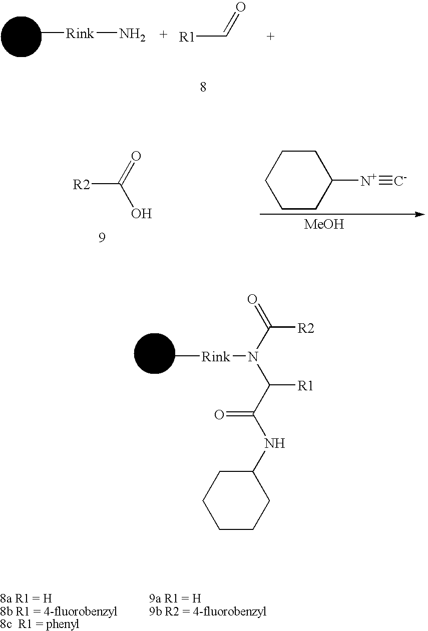

After Fmoc removal by 20% (v/v) piperidine/NMP, the ArgoGel-Rink-NH₂ resins (100 mg each, 0.36 mmole/g, 0.036 mmole) were treated sequentially with aldehyde (8), cyclohexyl isocyanide (45 μL, 0.36 mmole, 10 eq) and acid (9) in MeOH. The reactions were allowed to proceed for 5, 15, 45, 135, 330 min and overnight, respectively, before they were washed with MeOH (5×) and DCM (3×). A fixed amount of each resin was cleaved by 50% TFA/DCM for 20 min for analysis. The level of completion of each reaction was determined by peak height of UV trace of each product on LC/MS.

LC/MS result indicated that Ugi reaction of 0.2 equivalent of 8a (0.58 μL, 7.2×10⁻³ mmole) and 10 equivalent of 9b (50 mg, 0.36 mmole) at room temperature proceeded to completion after 135 min, while at 50° C. the reaction completed after 15 min.

LC/MS result indicated that Ugi reaction of 10 equivalent of 8b (39 μL, 0.36 mmole) and 0.2 equivalent of 9a (0.27 μL, 7.2×10⁻³ mmole) at room temperature proceeded to completion after 135 min, while at 50° C. the reaction completed after 15 min.

LC/MS result indicated that Ugi reaction of 0.2 equivalent of 8b (0.78 μL, 7.2×10⁻³ mmole) and 10 equivalent of 9b (50 mg, 0.36 mmole) at room temperature proceeded to completion after 135 min, while at 50° C. the reaction completed after 45 min.

LC/MS result indicated that Ugi reaction of 10 equivalent of 8b (39 μL, 0.36 mmole) and 0.2 equivalent of 9b (1.0 mg, 7.2×10⁻³ mmole) at room temperature proceeded to completion after 45 min, and the reaction also completed after 45 min at 50° C.

Stoichiometry Study of Ugi Reaction

After De-Fmoc, ArgoGel-Rink-NH resin (1.00 g, 0.36 mmole/g, 0.36 mmole) was treated sequentially with aldehyde (8), cyclohexyl isocyanide (448 μL, 3.6 mmole, 10 eq) and acid (9) in MeOH. The reaction was allowed to proceed at 50° C. for 1 hour before it was washed with MeOH (5×) and DCM (3×). The product on resin was cleaved by 50% TFA/DCM for 1 hour. The resin was filtered and washed with DCM (5×) and MeOH (5×). The filtrate and the washing were combined and concentrated. Final product was characterized by ¹H NMR and LC/MS. Yield of the reaction was determined by the weight of final product. In some cases below, the products were not pure, but could be purified by simple chromatography. The products were identified by LC-MS but the proportion of desired product in the crude material was not determined.

Products Prepared Using the Ugi Reaction

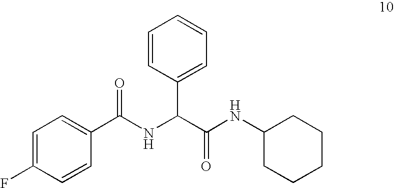

Ugi reaction of 10 equivalent of 8c (366 μL, 3.6 mmole) and 10 equivalent of 9b (504 mg, 3.6 mmole) gave 10 as off-white crystal (100 mg, 78.7%).

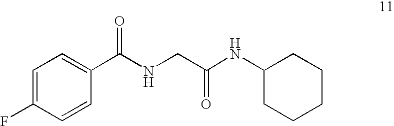

Ugi reaction of 0.2 equivalent of 8a (5.9 μL, 0.072 mmole) and 10 equivalent of 9b (504 mg, 3.6 mmole) gave 11 as off-white crystal (42.6 mg, 211% to theoretical yield).

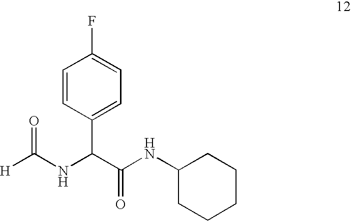

Ugi reaction of 10 equivalent of 8b (386 μL, 3.6 mmole) and 10 equivalent of 9a (2.7 μL, 0.072 mmole) gave 12 as off-white crystal (25.7 mg, 127% to theoretical yield).

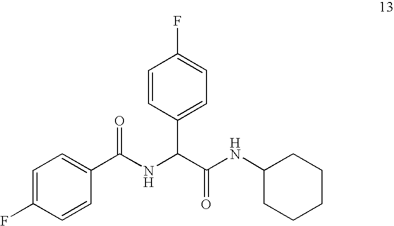

Ugi reaction of 0.2 equivalent of 8b (7.7μL, 0.072 mmole) and 10 equivalent of 9b (504 mg, 3.6 mmole) gave 13 as off-white crystal (43.4 mg, 164% to theoretical yield).

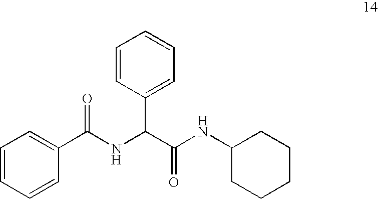

Ugi reaction of 10 equivalent of 8c (366 μL, 0.072 mmole) and 0.2 equivalent of 9b (10.1 mg, 0.072 mmole) gave 10 as off-white crystal (66.7 mg, 263% to theoretical yield). The possible byproduct is shown as 14.

### Example 6

This example illustrates the preparation of PET-ready compounds/library using a diketopiperazine template and a REM linker.

Preparation of Boc-Protected Dipeptides

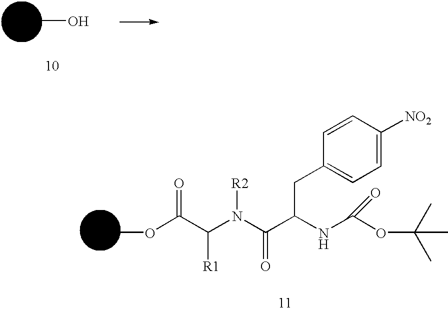

N-Boc-protected amino acid (see table below; 2.88 mmol, 12 eq) was dissolved in NMP (5 mL). To the resulting solution was added diisopropylcarbodiimide (DIC; 0.187 mL, 1.44 mmol, 6 eq) and DMAP (10 mg). After standing for 5 min the solution was added to ArgoGel-OH resin 10 (Argonaut Technologies, San Carlos, Calif.; 0.5 g, 0.46 mmol/g, 0.23 mmol, 1.0 eq.). The mixture was shaken for 3 h at 20° C. then drained and washed with NMP (3×10 mL) and DCM (3×10 mL). The resin was treated with a solution of trifluoroacetic acid (TFA) and DCM (1:1; 5 mL) and shaken for 20 min, then drained and washed with DCM (4×10 mL) and NMP (10 mL) to give amino acid resin.

4-Nitrophenylalanine (0.25 g, 0.8 mmol, 8 eq) was dissolved in NMP (2 mL). To the resulting solution was added HBTU (0.30 g, 0.8 mmol, 8 eq) and DIEA (0.272 mL, 1.6 mmol, 16 eq.). After standing for 5 min this was added to amino acid resin (0.186 g, 0.47 mmol/g, 0.1 mmol). The resulting mixture was shaken for 30 min. then drained and the resin 11 washed with NMP (3×10 mL) and DCM (3×10 mL). Completion of coupling was confirmed by ninhydrin test. The resin was treated with a solution of trifluoroacetic acid (TFA) and DCM (1:1; 5 mL) and shaken for 20 min, then drained and washed with DCM (4×10 mL) and NMP (10 mL) to give Boc-deprotected dipeptide resin 12.

Cyclization of Dipeptides to Diketopiperazines

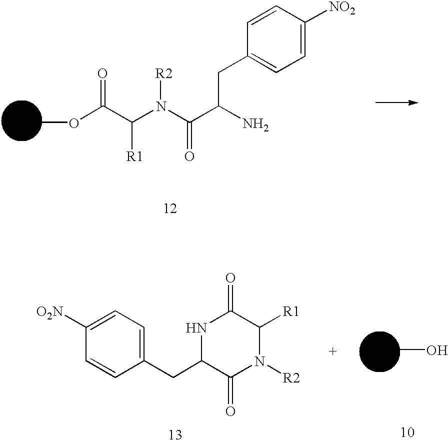

Dipeptide resin 12 (0.12 g, 0.47 mmol/g, 0.056 mmol) was treated with DCM (2.0 mL) and 2M ammonia in methanol (2.0 mL) and the resulting mixture shaken at 20° C. Portions of the reaction supernatant (0.45 mL) were removed after the following times: 5, 15, 45 min, 18 h. Each sample was evaporated to dryness under reduced pressure, and the residue dissolved in 50% aqueous acetonitrile (0.2 mL) and analyzed by LC-MS. The relative quantity of released diketopiperazine 13 at each time point was determined by the height of the peak with the correct mass in the LC-MS chromatogram. The t ½ for each resin was determined as the time at which half of the quantity released in the 18 h sample would have been released.

The cyclization experiment was repeated at −6° C.

The table below provides the T½ for the various cleavage reactions at 20° C. and at −6° C.

| Amino Acid | R1 | R2 | 20° C. | −6° C. |
|---|---|---|---|---|
| Glycine | H | H | 10 | 60 |
| Sarcosine | H | Me | <5 | <5 |
| Alanine | Me | H | 25 | 180 |
| N-Methyl Alanine | Me | Me | <5 | <5 |
| Proline | CH₂— | CH₂— | <5 | <5 |

It is understood that the examples and embodiments described herein are for illustrative purposes only and that various modifications or changes in light thereof will be suggested to persons skilled in the art and are to be included within the spirit and purview of this application and scope of the appended claims. One of skill in the art will also understand that all methods and applications of the invention related to PET-ready libraries can also be applied to making SPECT-ready and autoradiography-ready libraries. All publications, patents, and patent applications cited herein are hereby incorporated by reference in their entirety for all purposes.

What is claimed is:

1. A method for determining the distribution of an active agent in a tissue, said method comprising:
(a) screening a PET-ready library of potential agents against a biological target;
(b) identifying at least one of said potential agents as an active agent;
(c) preparing a PET-labeled version of said active agent, wherein said preparing comprises incorporating a PET-label into the final or penultimate step of active agent synthesis;
(d) administering said PET-labeled version of said active agent to a subject; and
(e) measuring the distribution of said active agent in at least one tissue of said subject.

2. A method in accordance with claim 1, wherein said PET-label is selected from the group consisting of ¹¹C, ¹⁸F, ⁷⁶Br, ¹²⁴I and ¹³N.

3. A method in accordance with claim 1, wherein said PET-ready library of potential agents is prepared using solid phase methods,

4. A method in accordance with claim 1, wherein said final or penultimate step of active agent synthesis is an alkylation step.

5. A method in accordance with claim 1, wherein said final or penultimate step of active agent synthesis is a methylation step.

6 A method in accordance with claim 1, wherein said final or penultimate step of active agent synthesis is an acetylation step.

7 A method in accordance with claim 1, wherein said PET-ready library comprises from about 12 to about 144 potential agents.

8. A method in accordance with claim 1, wherein said PET-ready library is prepared on a resin.

9. A method in accordance with claim 1, wherein said subject is selected from the group consisting of a mouse, rat, dog, cat, sheep, monkey and human.
